(19) United States
(12) Patent Application Publication
(10) Pub. No.: US 2026/0199123 A1
(43) Pub. Date: Jul. 16, 2026

(54) CONTRACEPTIVE

(71) Applicant: LUWI, LLC, Mt. Prospect, IL (US)

(72) Inventor: Lisa Kinsella, Mt. Prospect, IL (US)

(73) Assignee: LUWI, LLC, Mt. Prospect, IL (US)

(21) Appl. No.: 19/017,368

(22) Filed: Jan. 10, 2025

(51) Int. Cl. A61F 6/04

(52) U.S. Cl. CPC A61F 6/04

(57) ABSTRACT

One embodiment of a contraceptive may include a body or sheath including a first side and a second side connected to the first side along a seam that is heat-fused to bond the first and second sides together. The first and second sides of the body may be a polyurethane material, such as an aromatic polyether-based thermoplastic polyurethane. In an embodiment, the aromatic polyether-based thermoplastic polyurethane is composed of PT9511S produced by Covestro®. A retention member may be attached to an opening defined between the first and second sides of the body. The retention member may be formed of an aromatic polyether-based thermoplastic polyurethane. The aromatic polyether-based thermoplastic polyurethane may be composed of Texin RxT85A resin produced by Covestro. A method may be used to form the barrier method contraceptive.

### BACKGROUND

Barrier method contraception has become normalized in today's society to both (i) prevent pregnancy, and (ii) reduce or eliminate sexually transmitted diseases (STI's). The most common form of contraceptives is condoms for males. Such condoms are often uncomfortable due to being tight and difficult to put on. Moreover, condoms have a known failure rate for both pregnancies and STI's due to manufacturing defects, over-stretching, and general failures of the condom. The typical material of condoms is latex, such as natural rubber latex. Latex for condoms is often sufficiently thick to reduce or eliminate failures, but the thickness and material reduce sensitivity and heat transfer such both male and females lose desired sensation during intimacy. Still yet, there is a small, but statistically significant, percentage of the population, both males and females, that is allergic to latex, which results in those people avoiding latex condoms altogether. As a result of the above problems, people are often make risky decisions to remove or forgo usage of latex-based condoms. Moreover, because condoms are generally controlled by males, females in heterosexual relationships have to rely on males to purchase, use, and properly apply the condoms, which often leads to difficult situations for females. Accordingly, there is a need for a contraceptive that is more sensitive, has increased heat transfer, fits well, is easy to put on, and is not exclusive to males.

### BRIEF SUMMARY

To provide for an improved contraceptive that overcomes the shortcomings of condoms, the principles provided herein provide for a female contraceptive that is formed of a material, such as polyurethane, that provides increased comfort, heat transfer, and reliability, and is controlled by a “receiver” (e.g., female). For the purposes of this disclosure, it is understood that a receiver may be male and used anally, but is being described herein as a female contraceptive as a vast majority of users of the contraceptive are anticipated to be female. In an embodiment, the contraceptive may have a body or sheath that is shaped to have a narrower waist than a proximal end and distal end (e.g., an “hour-glass” shape). The proximal end defines an opening of the contraceptive, and may include a retention member positioned at an opening of the contraceptive that may be formed of the same or similar material as the sheath or sleeve of the contraceptive. The retention member may be circular or any other geometric shape and be heat-fused to the proximal end of the sheath of the contraceptive, whereby the sheath of the contraceptive may be partially or fully rolled around the retention member prior to being heat-fused. In being heat-fused, heat may be applied to cause the sheath and retention member to melt and fuse to one another.

One embodiment of a contraceptive may include a body or sheath including a first side and a second side connected to the first side along a seam that is heat-fused to bond the first and second sides together. The first and second sides of the sheath may be a polyurethane material, such as an aromatic polyether-based thermoplastic polyurethane. In an embodiment, the aromatic polyether-based thermoplastic polyurethane is composed of PT9511S produced by Covestro®. A retention member may be attached to an opening defined between the first and second sides of the sheath. The retention member may be formed of an aromatic polyether-based thermoplastic polyurethane. The aromatic polyether-based thermoplastic polyurethane may be composed of Texin RxT85A resin produced by Covestro.

One embodiment of a method of manufacturing a contraceptive may include placing a first sheet. A second sheet may be placed on the first sheet. A force may be applied to the first and second sheets. An outline defining a sheath of the contraceptive may be cut. A seam may be formed along the outline defining the sheath of the contraceptive by applying heat to the first and second sheets, where the heat may cause the seam to be heat-fused together.

### BRIEF DESCRIPTION OF THE FIGURES

Illustrative embodiments of the present invention are described in detail below with reference to the attached drawing figures, which are incorporated by reference herein and wherein:

FIGS. 1A-1B are illustrations of an illustrative contraceptive shown from different perspective angles;

FIGS. 2A-2G are illustrations of an illustrative contraceptive shown from different angles; and

FIGS. 3A and 3B are illustrative top and bottom views of a contraceptive 300 that may have the same or similar structure and materials as described with regard to FIGS. 1A, 1B, and 2A-2G.

### DETAILED DESCRIPTION

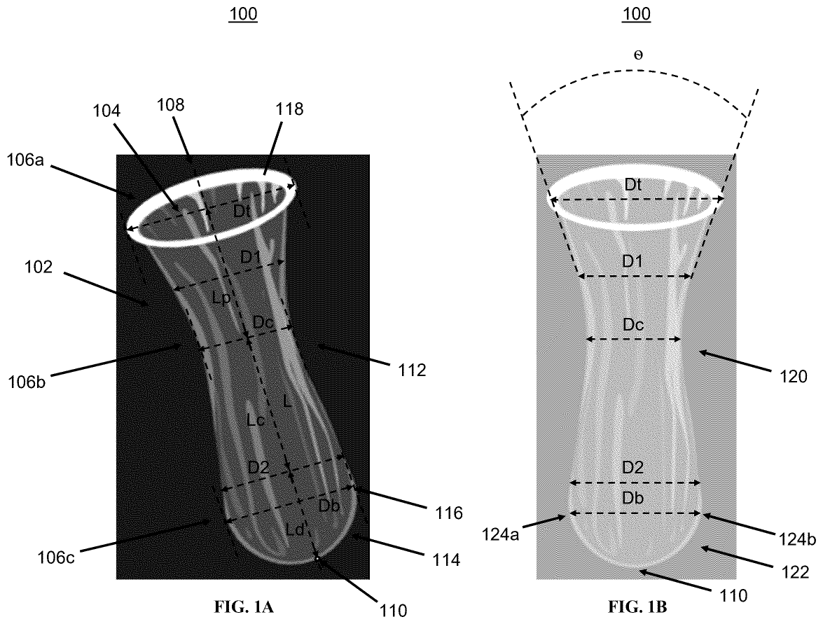

With regard to FIGS. 1A and 1B, illustrations of an illustrative contraceptive 100 at different perspective views is shown. The contraceptive 100 may include a body or sheath 102 formed by two sheets of film (see below). When the sheets of the sheath 102 are connected, as further described herein, an opening 104 may be defined at a proximal end 106a. The sheath 102 may further define a center portion 106b, and distal end 106c. The proximal end 106a may have a diameter Dt (top diameter), the center portion 106b may have a diameter Dc (center diameter), and distal end 106c may have a diameter Db (bottom diameter). The diameters Dt, Dc, and Db may be defined by lines normal to a central axis 108 of the contraceptive 100. The center portion 106b may define a waist having the shortest diameter Dc that is at a center location between a tip 110 through which the central axis 108 extends or may be positioned closer to the proximal end 106a or distal end 106c. An outer profile of the sheath 102 of the contraceptive 100 may form a concave shape 112 that transitions to a convex shape 114 at a plane 116, which may coincide with the diameter Db being the largest. From the plane 116, the sheath tapers in a curved manner to the tip 110.

In an embodiment, overall length L of the contraceptive 100 extends from the opening 104 to the tip 110. The overall length L may be 180 mm in an embodiment. Alternative lengths may be utilized, as further described herein. A length at the distal end Ld defined by a location at which a circular profile at the distal end 106c intersects with a curved profile of the center portion 106b, such as shown where diameter D2 is located, may be 52 mm. A length of the center portion 106b may be defined by the curved profile that extends from the diameter D2 is located to the proximal end 106a at which the profile of the contraceptive 100 may be linear at diameter D1 and extends at an opening angle from the curved profile of the center portion 106b. The opening angle θ that defines an angle of the sheath as it extends from the center portion 106b to the proximal end 106a. In an embodiment, the opening angle θ may be 42 degrees such that each opposing opening wall may be 21 degrees. It should be understood that alternative angles and dimensions may be utilized in accordance with the principles described herein.

The diameter D1 may define a starting location for an arc or curve 120 of a surface profile of the contraceptive 100 that extends across diameter Dc, and the diameter D2 may define an ending location for the arc 120 (e.g., parabola) of the surface profile of the contraceptive 100. As described above, the diameters D1 and D2 define locations along the surfaces of the sheath 102 that define the distal end 106c, center portion 106b, and proximal end 106a that are themselves defined by different geometric shapes (e.g., circle, curve, and straight line). A curve 122 extending from opposite points 124a and 124b (collectively 124) of the sheath along a plane that is perpendicular to the central axis 108 at diameter Db (i.e., where a concave curve or straight portion transitions to a convex curve that extends across the tip 110 may have a circular profile. It should be understood that a non-circular profile (e.g., oval) between the opposite points 124 and through the tip 110 may be utilized, as well. The curve of the surface of the contraceptive 100 along the center portion 106b (i.e., extending between D1 and D2) may be 139.52 mm (5.493 in), where, in this embodiment, the diameter of the contraceptive 100 at D1 is 84.52 mm (3.328 in) and the diameter of the contraceptive 100 at D2 is 77.61 mm (3.056 in). It should be understood that alternative dimensions may be utilized. For example, in an alternative embodiment, the center diameter Dc may be 68 mm, bottom diameter Db may be 82.5 mm, and top diameter Dt may be 81.5 mm.

In an embodiment, a standard or average contraceptive may have the approximate dimensions listed in TABLE I. In being approximate, the dimensions may deviate by a small percentage, such as one percent or less. The dimensions in the range column may be used for alternative sizes, such as small and large, of contraceptives (e.g., up to 10% above and below the standard dimensions). One or more of the dimensions may be varied from those shown.

TABLE I
Illustrative Dimensions

| Parameter | Dimension (Standard) | | Range |
|---|---|---|---|
| Overall Length (L) | 180 mm | (7.087 in) | 162 mm-198 mm |
| Distal Length (Ld) | 52 mm | (2.047 in) | 46.8 mm-57.2 mm |
| Center Length (Lc) | 90 mm | (3.543 in) | 81 mm-99 mm |
| Proximal | 38 mm | (1.496 in) | 34.2 mm-41.8 mm |
| Length (Lp) | | | |
| Bottom | 81 mm | (3.189 in) | 72.9 mm-89.1 mm |
| Diameter (Db) | | | |
| Center | 65.95 mm | (2.597 in) | 59.36 mm-72.55 mm |
| Diameter (Dc) | | | |
| Top Diameter (Dt) | 110.06 mm | (4.333 in) | 99.05 mm-121.07 mm |
| Diameter D1 | 84.52 mm | (3.328 in) | 76.07 mm-92.97 mm |
| Diameter D2 | 77.61 | mm | 69.85 mm-85.37 mm |
| Opening Angle ( [symbol] ) | 42 | degrees | 37.8 degs-46.2 degs |

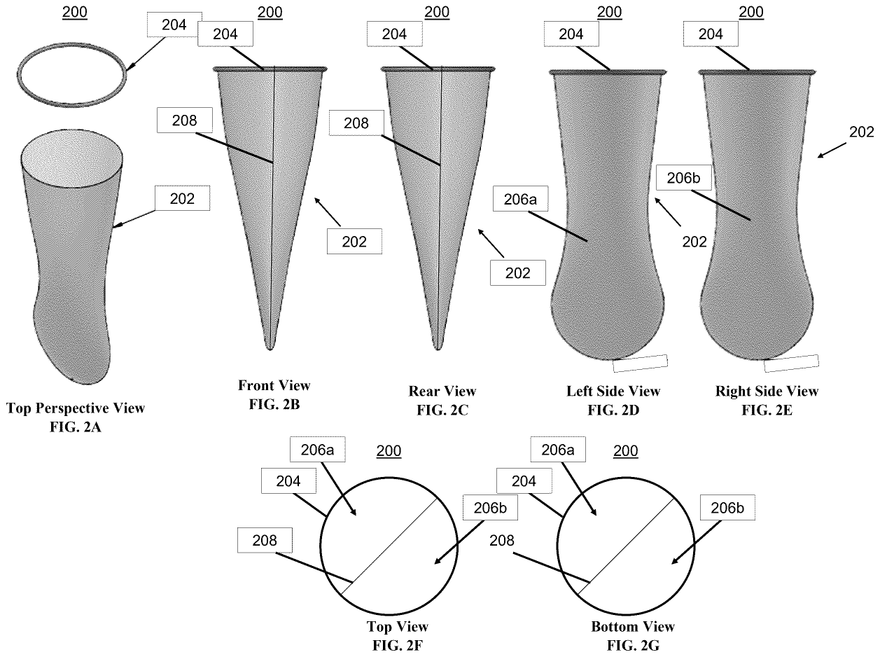

The sheath 102 may be formed of two sheets of thermoplastic film having matching sizes and shapes. The thermoplastic film sheets may be placed together and pressed, heat-fused to form a seam, and cut heat-fused together along the seam that defines an outer profile shape of the sheath 102. After the sheath 102 composed of the two sheets is formed, the two sheets may lay flat with the shape, but when used, and if force is pressed evenly throughout the sheath 102, the sheath 102 would be substantially circular in a cross-sectional view (e.g., slight bending exists at the seams, but otherwise substantially circular due to the shape at least with regard to the center portion 106b and top portion 106a. Because the heat-fused portion of the seam along the edge is on the outside of the cavity defined by the two sheets, a user does not contact the seam itself, so no contact irritation results from use of the contraceptive 100. The distal end 106c, as it tapers, may be less circular and begin having more of an oblong shape as the tapering transitions towards a “V” shape towards the distal end 106c (see FIGS. 2B and 2C, for example). The material of the sheets of the sheath 102 may be a polyurethane material. In an embodiment, the polyurethane is an aromatic polyether-based thermoplastic polyurethane. The aromatic polyether-based thermoplastic polyurethane may be PT9511S produced by Covestro®. It should be understood that the aromatic polyether-based thermoplastic polyurethane may be any alternative compound produced by Covestro or any other manufacture having the same or similar physical and chemical properties. In an embodiment, the retention member is formed of an aromatic polyether-based thermoplastic polyurethane.

Defining the opening may include a retention member 118 that may be circular (or other shape) that is connected to the sheath 102. The retention member 118 may be flexible in that it can be squeezed towards the central axis 108, but retain a circular shape when forces are not applied thereto. In an embodiment, the retention member 118 may be formed of the same or similar material as the sheath 102. The retention member 118 and sheath 102 may be connected to one another using a heat-fusion process, whereby the retention member 118 may be contacted with a proximal end of the sheath 102 may heated to the point that the materials of the retention member 118 and sheath 102 melt and fuse together. The temperature may be about 240 degrees Fahrenheit (F) to about 250 F. In being about 240 and 250 degrees Fahrenheit, the temperature may be a few degrees higher or lower. It should be understood that the temperature may be varied depending on time and pressure being applied. In an embodiment, pressure and heat may be simultaneously applied during the heat fusion process. For example, the time may be about 3 seconds and pressure may be about 30+/−5 pounds per square inch (PSI). In an embodiment, the end of the sheath 102 may be wrapped over and pressed against the retention member 118 prior to the heat-fusion process, thereby ensuring that the retention member 118 fully heat-fused with the sheath 102 along the entire opening 104 to avoid leakage, separation, or other failure.

The retention member 118 may be sized with a large enough diameter to be retained against the vulva or anal cavity so as to not invaginate or be pulled into the anal cavity, respectively. In an embodiment, another retention member (not shown) that is smaller in diameter, if circular, than the retention member 118 may be placed within the sheath 102 and be used to engage a woman's cervix so as to maintain position of the contraceptive 100. Use of another ring may allow for a woman to maintain the contraceptive for relatively long periods of time, such as up to 8 hours. Use of the contraceptive 100 without an inner ring makes the contraceptive more of an “at the moment” usage contraceptive. The contraceptive 100 may be inserted into a sheath cavity or may be applied to a penis or insertion device. Lubrication would also be utilized as part of the insertion process. In an embodiment, the contraceptive 100 may be packaged with lubrication or lubrication may be added during use.

With regard to FIGS. 2A-2G, illustrations of an illustrative contraceptive 200 at different angles are shown. The contraceptive 200 is shown in a top-perspective view with a sheath 202 being separated from a retention member 204. The retention member 204, of course, is heat-fused to the body 202. The body 202 may be formed of two sheets 206a and 206b (collectively 206) that are heat-fused along a seam 208 that extends along an outer edge around the sheets 206 during manufacturing. The heat that heat-fuses the two sheets 206 together may be at a temperature between about 260 degrees Fahrenheit to about 270 degrees Fahrenheit so as to be sufficiently hot to melt the two sheets 206 together, but not cause burning or over-melting that would result in damaging the seam. In an embodiment, pressure and heat may be simultaneously applied to the two sheets during the heat fusion process. For example, the time may be about 4 seconds and pressure may be about 30+/−5 PSI. The seam 208 extends around the two sheets 206 during the production process as a press, heat, and cut process that defines the shape of the two sheets is formed simultaneously on the entire two sheets 206 that produce the sheath 202 of the contraceptive 200.

In operation, the two sheets 206 may be connected prior to the retention member 204 be connected thereto. An excess portion of the two sheets 206 that extend past the retention member 204 from the sheath 202 may be removed such that the retention member 204 defines the opening of the contraceptive 200. The features and materials of the contraceptive 200 may be the same or similar to those describe with regard to the contraceptive 100 of FIGS. 1A and 1B. The contraceptive of FIGS. 2A-2G further provide for an ornamental appearance of a contraceptive, and it should be understood that alternative shapes and dimensions may be utilized in accordance with the principles described herein. It should be understood that the sheets 206 may be transparent, translucent, and/or opaque.

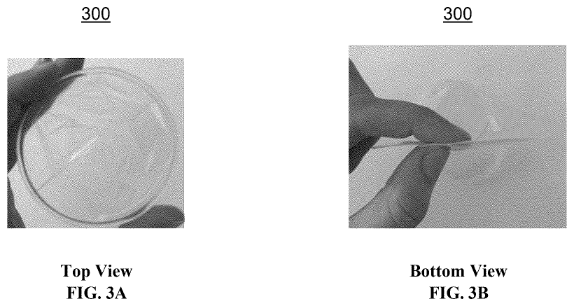

With regard to FIGS. 3A and 3B, illustrative top and bottom views of a contraceptive 300 that may have the same or similar structure and materials as described with regard to FIGS. 1A, 1B, and 2A-2G are shown.

### Features

A contraceptive including a body or sheath that includes a first side, and a second side connected to the first side along a seam that is heat-fused to bond the first and second sides together.

The seam may include a portion from each of the first and second sides having been pressed and melted together and extends outwards from an outside surface of the sheath. The seam is approximately the same thickness as the first and second sides. The first side and the second side may be formed of a polyurethane material. The polyurethane material may be an aromatic polyether-based thermoplastic polyurethane. The aromatic polyether-based thermoplastic polyurethane may be PT9511S produced by Covestro®. A retention member may be attached to an opening defined between the first and second sides of the sheath, and where the retention member may be formed of an aromatic polyether-based thermoplastic polyurethane. The aromatic polyether-based thermoplastic polyurethane may be Texin RxT85A resin produced by Covestro®. The sheath may have a larger diameters towards the proximal and distal ends than a center portion.

A diameter of an opening defined by the sheath may be approximately 110 mm (4.333 in). A center diameter defining a minimum diameter of the sheath may be approximately 65.95 mm. A bottom diameter may be approximately 81 mm. An arc having a minimum diameter of the sheath may have a curvature starting at approximately 84.52 mm on one side of a center diameter of 66.95 mm at a minimum diameter of the sheath and ending at approximately 77.61 mm on an opposite side of the center diameter, where the starting and ending diameters are positioned at locations prior to the shape of the sheath being substantially linear or transition to a convex curve.

A length of the contraceptive may be approximately 180 mm (7.087 in). An opening angle θ may be approximately 42 degrees. A center portion of a surface of the sheath may have a concave shape, and a distal end opposite an opening may have a convex shape that has a diameter larger than a minimum of the convex shape.

One embodiment of a method of manufacturing a contraceptive may include placing a first sheet. A second sheet may be placed on the first sheet. A force may be applied to the first and second sheets. An outline defining a sheath of the contraceptive may be cut. A seam along the outline defining the sheath of the contraceptive by applying heat to the first and second sheets may be formed. The heat may cause the seam to be heat-fused together.

A retention member along an opening at a proximal end of the sheath may be contacted. Heat may be applied to the sheath and retention member to heat-fuse the sheath and retention member to one another. Placing the first and second sheets may include placing first and second sheets formed of polyurethane material.

The process may further include contacting a retention member along an opening at a proximal end of the sheath, where the retention member is formed of polyurethane material. A seam may be heat-fused between the first and second sheets. The retention member may be heat-fused to the first and second sheets at a proximal end that defines an opening of the sheath.

The illustrations included herewith are not meant to be actual views of any particular device, but are merely idealized representations that are employed to describe embodiments herein. Elements and features common between figures may retain the same numerical designation except that, for ease of following the description, for the most part, reference numerals begin with the number of the drawing on which the elements are introduced or most fully described. In addition, the elements illustrated in the figures are schematic in nature.

As used herein, the singular forms “a,” “an,” and “the” are intended to include the plural forms as well, unless the context clearly indicates otherwise.

As used herein, “or” includes any and all combinations of one or more of the associated listed items in both, the conjunctive and disjunctive senses. Any intended descriptions of the “exclusive-or” relationship will be specifically called out.

As used herein, the term “configured” refers to a structural arrangement such as size, shape, material composition, physical construction, logical construction or other operative arrangement of at least one structure and at least one apparatus facilitating the operation thereof in a defined way (e.g., to carry out a specific function or set of functions).

As used herein, the phrases “coupled to” or “coupled with” refer to structures operably connected with each other, such as connected through a direct connection or through an indirect connection (e.g., via another structure or component).

The foregoing method descriptions and/or any process flow diagrams are provided merely as illustrative examples and are not intended to require or imply that the steps of the various embodiments must be performed in the order presented. As will be appreciated by one of skill in the art, the steps in the foregoing embodiments may be performed in any order. Words such as “then,” “next,” etc. are not intended to limit the order of the steps; these words are simply used to guide the reader through the description of the methods. Although process flow diagrams may describe the operations as a sequential process, many of the operations may be performed in parallel or concurrently. In addition, the order of the operations may be re-arranged. A process may correspond to a method, a function, a procedure, a subroutine, a subprogram, etc. When a process corresponds to a function, its termination may correspond to a return of the function to the calling function or the main function.

The previous description is of various preferred embodiments for implementing the disclosure, and the scope of the invention should not necessarily be limited by this description. The scope of the present invention is instead defined by the claims.

1. A contraceptive, comprising:
a sheath having a proximal end and a distal end, including:
a first side having an edge with a profile defining an hour-glass shape being a continuous curve between a first diameter and a second diameter; and
a second side having an edge with a reciprocal hour-glass shape as the first side, and connected to the first side along the edges that define a seam that is heat-fused to bond the first and second sides together from the proximal end to the distal end.

2. The contraceptive according to claim 1, wherein the seam that is heat-fused includes a portion from each of the first and second sides having been pressed and melted together and extends outwards from an outside surface of the sheath.

3. The contraceptive according to claim 2, wherein the seam is approximately the same thickness as the first and second sides.

4. The contraceptive according to claim 1, wherein the first side and the second side are formed of a polyurethane material.

5. The contraceptive according to claim 4, wherein the polyurethane material is an aromatic polyether-based thermoplastic polyurethane.

6. The contraceptive according to claim 5, wherein the aromatic polyether-based thermoplastic polyurethane is produced by Covestro®.

7. The contraceptive according to claim 5, further comprising a retention member attached to an opening located at the proximal end of the first and second sides of the sheath, and wherein the retention member is formed of an aromatic polyether-based thermoplastic polyurethane.

8. The contraceptive according to claim 7, wherein the aromatic polyether-based thermoplastic polyurethane is resin produced by Covestro®.

9. (canceled)

10. The contraceptive according to claim 1, wherein an opening defined by first and second sides at the proximal end is circular with a diameter of approximately 70.06 mm (2.76 in).

11. The contraceptive according to claim 1, wherein a center diameter of the hour-glass shape defining a minimum diameter of the sheath is approximately 41.99 mm (1.65 in).

12. The contraceptive according to claim 11, wherein a bottom diameter positioned between the second diameter and the distal end is approximately 51.57 mm (2.03 in), which is larger than the second diameter.

13. The contraceptive according to claim 1, wherein the sheath includes an arc having a minimum diameter of the sheath has the continuous curved shape starting at approximately 53.81 mm (2.12 in) on one side of a center diameter of 41.99 mm (1.65 in) at a minimum diameter of the sheath and ending at approximately 49.41 mm (1.95 in) on an opposite side of the center diameter, wherein the starting and ending diameters are positioned at locations prior to the shape of the sheath being substantially linear or transition to a convex curve.

14. The contraceptive according to claim 1, wherein a length of the contraceptive is approximately 180 mm (7.087 in).

15. The contraceptive according to claim 1, wherein an opening angle θ extending from the opening into and along the sheath is approximately 42 degrees.

16. (canceled)

17. A method of manufacturing a contraceptive, comprising:
placing a first sheet;
placing a second sheet on the first sheet;
applying a force to the first and second sheets;
cutting an outline, in the first and second sheets, to define-a sheath of the contraceptive, the outline defining edges of the first and second sheets, the edges of the sheath defining an hour-glass shape and having a continuous curve between a first diameter and a second diameter of the sheath; and
forming a seam along the edges of the sheath of the contraceptive by applying heat to the first and second sheets, the heat causing the seam to be heat-fused together.

18. The method according to claim 17, further comprising:
contacting a retention member along an opening at a proximal end of the sheath; and
applying heat to the sheath and retention member to heat-fuse the sheath and retention member to one another.

19. (canceled)

20. The method according to claim 17, wherein placing the first and second sheets includes placing the first and second sheets formed of polyurethane material; and further comprising:
contacting a retention member along an opening at a proximal end of the sheath, wherein the retention member is formed of polyurethane material; and
heat fusing the retention member to the first and second sheets at a proximal end that defines an opening of the sheath.

21. The contraceptive according to claim 1, wherein the hour-glass shapes includes a convex shape that extends from a first diameter positioned between the proximal end and a center diameter to a second diameter positioned between the center diameter and a bottom diameter.

22. The contraceptive according to claim 21, wherein:
diameter of the sheath (i) linearly grows larger than or remains the same from the second diameter to the bottom diameter, (ii) linearly gets smaller from the bottom diameter to the distal end, the continuous curved shape of the sheath being convex from the bottom diameter to the distal end of the sheath; and
diameter of the sheath (i) linearly grows larger or remains the same from the first diameter to the proximal end.

23. The contraceptive according to claim 22, wherein the diameter of the sheath linearly grows between the first diameter to an opening with an opening angle θ.

24. The contraceptive according to claim 1, wherein the sheath transitions from a concave shape of the hour-glass shape to a convex shape at the second diameter, the convex shape extending from the second diameter to the distal end of the sheath.

25. The contraceptive according to claim 1, wherein the contraceptive is independent of an inner ring.
